(19) United States
(12) Patent Application Publication
(10) Pub. No.: US 2007/0128688 A1
(43) Pub. Date: Jun. 7, 2007

(54) Method for cell-free protein synthesis using complementary oligonucleotide

(75) Inventors: Masamitsu Shikata, Kyoto (JP); Shinichiro Kobayashi, Kyoto (JP)

Correspondence Address: RADER FISHMAN & GRAUER PLLC, LION BUILDING, 1233 20TH STREET N.W., SUITE 501, WASHINGTON, DC, 20036, US

(73) Assignee: SHIMADZU CORPORATION, Kyoto (JP)

(21) Appl. No.: 11/604,334

(22) Filed: Nov. 27, 2006

(30) Foreign Application Priority Data
Dec. 7, 2005 (JP) 2005-352874

(51) Int. Cl. C12P 21/06; C12N 5/06

(52) U.S. Cl. 435/68.1; 435/348

(57) ABSTRACT

The present invention provides a practical cell-free protein synthesis method capable of easily synthesizing a large amount of protein at low cost. A method for cell-free protein synthesis performed in a reaction solution containing mRNA and a living cell-derived extract solution, the reaction solution containing an oligonucleotide complementary to a sequence present in a 3′ terminal region of the mRNA.

### BACKGROUND OF THE INVENTION

1. Field of the Invention

The present invention relates to a cell-free protein synthesis method. More specifically, the present invention relates to a synthesis method capable of easily increasing the amount of synthesized protein per unit time.

2. Disclosure of the Related Art

In cell-free protein synthesis technology, various factors have influence on the amount of synthesized protein. Among them, energy exhaustion and degradation of mRNA have great influence on the amount of synthesized protein.

In order to overcome the problem of energy exhaustion, for example, A. S. Spirin et al. have proposed a method in which a reaction solution for cell-free protein synthesis is fed into an ultrafiltration system, and then a solution containing amino acid, ATP, and GTP is continuously fed into the reaction solution by using a pump while degradation products of substrates and reaction products containing a synthesized protein are removed from the reaction solution. This method made it possible to maintain cell-free protein synthesis for a dozen or so hours, and as a result the amount of synthesized protein was more than 20 times higher than ever before (A. S. Spirin et al., Science. 242, 1162-1164 (1988), Japanese Examined Patent Publication No. H7-110236).

On the other hand, it is believed that the degradation of mRNA is caused by removal of a 5′ terminal cap structure of mRNA (i.e., decapping reaction) or degradation of a 3′ terminal poly(A) structure of mRNA (Microbiol Rev. 1996 Mar; 60(1); 233-49). This document describes that when mRNA is shortened to some extent by degradation of poly(A), decapping reaction occurs and then mRNA is rapidly degraded by a 5′ exonuclease.

In order to overcome the problem of degradation of mRNA in cell-free protein synthesis, a method using a ribonuclease inhibitor has been proposed. Further, a method for increasing the amount of synthesized protein by allowing the 3′ end of mRNA serving as a template in cell-free protein synthesis to have a loop structure has been proposed (Biotechnol Bioeng. 1999 Jul. 20; 64(2): 194-9).

### SUMMARY OF THE INVENTION

The ribonuclease inhibitor has an effect of inhibiting the activity of an endonuclease such as RNase A that can cleave the internal site of mRNA, but cannot inhibit the activity of an exonuclease that can cleave the 5′ or 3′ end of mRNA.

It is known that the addition of a cap structure after mRNA synthesis is useful for cell-free protein synthesis technology. However, addition of a cap structure is expensive and requires complicated pretreatment, and therefore from a practical viewpoint, addition of a cap structure is hardly carried out in the current method for cell-free protein synthesis. In most cases, cell-free protein synthesis is usually carried out by adding only an inhibitor.

The method using looped mRNA obtained by the ligation of the 5′ and 3′ ends thereof together requires effort because it is necessary to prepare a template DNA designed to allow such looped mRNA to be obtained.

Therefore, these technologies for suppressing the degradation of mRNA are not generally used in current cell-free protein synthesis technology. Even if these technologies are used, they are not put to practical use.

It is therefore an object of the present invention to provide a practical cell-free protein synthesis method capable of easily synthesizing a large amount of protein at low cost.

The present invention comprises the followings:

(1) A method for cell-free protein synthesis performed in a reaction solution containing mRNA and a living cell-derived extract solution, the reaction solution containing an oligonucleotide complementary to a sequence present in a 3′ terminal region of the mRNA;

(2) The method for cell-free protein synthesis according to (1), wherein the reaction solution further contains an oligonucleotide complementary to a sequence present in a 5′ terminal region of the mRNA;

(3) The method for cell-free protein synthesis according to (1) or (2), wherein the oligonucleotide is selected from the group consisting of DNA, DNA alanogues, RNA, and RNA analogues;

(4) The method for cell-free protein synthesis according to any of (1) to (3), wherein the sequence present in the 3′ terminal region of the mRNA is a poly(A) chain sequence;

(5) The method for cell-free protein synthesis according to any of (1) to (4), wherein the mRNA has a translation enhancer sequence at a position adjacent to a downstream side of the 5′ terminal region;

(6) The method for cell-free protein synthesis according to any of (1) to (5), wherein the oligonucleotide has a length of 15 to 40 bases;

(7) The method for cell-free protein synthesis according to any of (1) to (6), wherein a concentration of the oligonucleotide contained in the reaction solution is in a range of 0.1 to 2 μM; and

(8) The method for cell-free protein synthesis according to any of (1) to (7), wherein the living cell is an insect cell.

According to the present invention, it is possible to provide a practical cell-free protein synthesis method capable of easily synthesizing a large amount of protein at low cost.

### BRIEF DESCRIPTION OF THE DRAWINGS

FIG. 1 is a graph showing a result of protein synthesis by a conventional method (Control), a result of protein synthesis by a reference method (ASO1), and results of protein synthesis by methods of the present invention (“ASO2” and “ASO1+ASO2”).

### DETAILED DESCRIPTION OF THE INVENTION

The present invention provides a cell-free protein synthesis method. More specifically, the present invention provides a method for synthesizing a protein by using an oligonucleotide complementary to a sequence present in a terminal region of mRNA. In this specification, the term “protein” includes oligopeptide and polypeptide.

As a reaction solution to be used for cell-free protein synthesis, conventionally known ones may be basically used without any particular limitation. Generally, an aqueous solution containing at least a living cell-derived extract solution, potassium salt, magnesium salt, DTT (dithiothreitol), adenosine triphosphate, guanosine triphosphate, creatine phosphate, creatine kinase, amino acid, RNase inhibitor, tRNA, mRNA, and buffer is used as a reaction solution for cell-free protein synthesis.

Among them, as a living cell-derived extract solution, conventionally known ones may be used without any particular limitation. Particulary, insect cell-derived extract solutions are preferably used. As insect cells to be used for preparing such an insect cell-derived extract solution, High Five cells(manufactured by Invitrogen) derived from Trichoplusia ni egg cells or Sf21 cells (manufactured by Invitrogen) derived from Spodoptera fruglperda ovary cells are preferably used because they have high protein synthesis ability and can be cultured in a serum-free medium.

A method for preparing an insect cell-derived extract solution to be preferably used for a cell-free protein synthesis system according to the present invention is not particularly limited. For example, a method disclosed in Japanese Unexamined Patent Publication No. 2004-215651, that is, a method in which insect cells suspended in a solution for extraction are rapidly frozen and then the frozen cells are ruptured for extraction can be used. This method is preferably used for the following reasons: cell rupture is carried out under mild conditions and therefore components essential for cell-free protein synthesis can be taken out from the cells without damage; contamination with RNase and the like from tools and the like can be prevented; and incorporation of a substance inhibiting translation reaction, which is a concern in a case of cell rupture using a reagent such as a surfactant, can be avoided.

More specifically, the insect cell-derived extract solution is prepared in the form of an aqueous solution containing 1 mg/mL to 200 mg/mL, preferably 10 mg/mL to 100 mg/mL of a protein in a protein concentration, together with 10 mM to 500 mM, preferably 50 mM to 300 mM of potassium acetate, 0.1 mM to 10 mM, preferably 0.5 mM to 5 mM of magnesium acetate, 0.1 mM to 10 mM, preferably 0.5 mM to 5 mM of DTT, 1 μM to 50 mM, preferably 0.01 mM to 5 mM of PMSF, and 5 mM to 200 mM, preferably 10 mM to 100 mM of HEPES-KOH (pH 4 to 10, preferably 6.5 to 8.5). Preferably, such an insect cell-derived extract solution is subjected to nuclease treatment.

The reaction solution for cell-free protein synthesis is preferably prepared in such a manner that the insect cell-derived extract solution is contained in a proportion of 10 (v/v) % to 80 (v/v) %, particularly 30 (v/v) % to 60(v/v) %. In other words, the reaction solution for cell-free protein synthesis is preferably prepared in such a manner that the amount of an insect cell-derived extract contained in the entire reaction solution is 0.1 mg/mL to 160 mg/mL, more-preferably 3 mg/mL to 60 mg/mL in a protein concentration. If the amount of an insect cell-derived extract contained in the entire reaction solution is less than 0.1 mg/mL or exceeds 160 mg/mL in a protein concentration, there is a case where a synthesis rate of a target protein may be lower.

Components other than the extract solution contained in the reaction solution for cell-free protein synthesis, such as potassium salt, magnesium salt, DTT, adenosine triphosphate, guanosine triphosphate, creatine phosphate, creatine kinase, amino acid component, RNase inhibitor, tRNA, exogenous mRNA, and buffer may be appropriately determined by those skilled in the art. For example, the reaction solution for cell-free protein synthesis is preferably prepared as an aqueous solution containing 50 mM to 150 mM of potassium acetate, 0.5 mM to 3 mM of magnesium acetate, 0.2 mM to 5 mM of DTT, 0.1 mM to 5 mM of ATP, 0.05 mM to 5 mM of GTP, 10 mM to 100 mM of creatine phosphate, 10 μg/mL to 500 μg/mL of creatine kinase, 10 μM to 200 μM of amino acid component, 1 U/μL to 10 U/μL of RNase inhibitor, 10 μg/mL to 500 μg/mL of tRNA, 20 μg/mL to 1,000 μg/mL of exogenous mRNA, and 10 mM to 50 mM of HEPES-KOH (pH 6.5 to 8.5). More preferably, such a reaction solution for cell-free protein synthesis further contains 0.1 mM to 10 mM of EGTA.

In the present invention, an oligonucleotide satisfying a certain condition is added to the reaction solution for cell-free protein synthesis.

The oligonucleotide satisfying a certain condition is one complementary to a sequence present in a terminal region of mRNA contained in the reaction solution for cell-free protein synthesis. More specifically, at least an oligonucleotide complementary to a sequence present in a 3′ terminal region of the mRNA is contained in the reaction solution for cell-free protein synthesis. Further, an oligonucleotide complementary to a sequence present in a 5′ terminal region of the mRNA may also be contained in the reaction solution for cell-free protein synthesis.

In the present invention, the term “terminal region” refers to a region having no influence on protein synthesis, that is, a region other than a region involved in protein synthesis (hereinafter, the term “region involved in protein synthesis” will be also referred to as a “translation-related region”) in an untranslated region of mRNA. Such terminal regions are present on upstream and downstream sides of the translation-related region. Hereinafter, the terminal region present on the upstream side of the translation-related region will be referred to as a “5′ terminal region”, and the terminal region present on the downstream side of the translation-related region will be referred to as a “3′ terminal region”. These terminal regions will be described in more detail below.

The translation-related region contains a coding region from an initiation codon to a termination codon, and may further contain an untranslated region comprising a sequence involved in binding of ribosomes or a translation enhancer sequence. Examples of a sequence involved in the binding of ribosomes include a kozak consensus sequence and a Shine-Dalgarno sequence. An example of a translation enhancer sequence includes an Ω sequence. In the present invention, the translation-related region of mRNA preferably contains a translation enhancer sequence.

In the present invention, the 5′ terminal region refers to a region (comprising continuous bases) from a 5′ terminal base to a base adjacent to the upstream side of the translation-related region in mRNA. More specifically, the 5′ terminal region refers to a region from a 5′-terminal base to a base adjacent to the upstream side of an initiation codon in mRNA; a region from a 5′ terminal base to a base adjacent to the upstream side of a ribosome binding sequence in mRNA; or a region from a 5′ terminal base to a base adjacent to the upstream side of a translation enhancer sequence in mRNA. In the present invention, it is possible to synthesize a large amount of protein without adding a cap structure to mRNA, but in a case where mRNA has a cap structure (m⁷G-(5′)pppX(m)pY(m)p- or m⁷G-(5′)pppX-), the base X in the cap structure is defined as a 5′ terminal base.

The sequence present in the 5′ terminal region refers to the entire base sequence of the 5′ terminal region (i.e., a sequence from a 5′ terminal base to a base adjacent to the upstream side of an initiation codon in mRNA; a sequence from a 5′ terminal base to a base adjacent to the upstream side of a ribosome binding sequence in mRNA; or a sequence from a 5′ terminal base to a base adjacent to the upstream side of a translation enhancer sequence in mRNA) or a partial base sequence selected from the 5′ terminal region so as to have any given length.

Similarly, in the present invention, the 3′ terminal region refers to a region (comprising continuous bases) from a base adjacent to the downstream side of the translation-related region to a 3′ terminal base in mRNA. More specifically, the 3′ terminal region refers to a region from a base adjacent to the downstream side of a termination codon to a 3′ terminal base in mRNA. Such a region may contain a poly(A) signal sequence or a poly(A) chain sequence. Here, the term “poly(A)” refers to polyadenylic acid or an oligomer of adenylic acid.

The sequence present in the 3′ terminal region refers to the entire base sequence of the 3′ terminal region (i.e., a sequence from a base adjacent to the downstream of a termination codon to a 3′ terminal base in mRNA) or a partial base sequence selected from the 3′ terminal region so as to have any given length. In the present invention, a poly(A) chain sequence is preferably selected as the sequence present in the 3′ terminal region.

The sequence present in the 5′ terminal region and the sequence present in the 3′ terminal region (hereinafter, the term “sequence present in the terminal region” will be also referred to as a “terminal side sequence”) preferably have a length of 15 to 40 bases, more preferably a length of 20 to 30 bases. In a case where the terminal region has a base length longer than such a preferred base length, a partial sequence having the preferred base length can be selected as a terminal side sequence from the terminal region. It is to be noted that partial sequences having different base lengths can also be selected as terminal side sequences from one terminal region.

As an oligonucleotide to be used in the present invention, that is, the oligonucleotide complementary to the terminal side sequence, at least one is selected from the group consisting of DNA, DNA analogues, RNA, and RNA analogues (hereinafter, DNA analogues and RNA analogues will also be referred to as “nucleic acid analogues”).

The DNA analogues are not particularly limited as long as they can be complementarily bound to the terminal side sequence. More specifically, the DNA analogues may be used without any particular limitation as long as they are obtained by modifying a phosphate main chain, deoxyribose, and/or a base selected from adenine, guanine, cytosine, and thymine of DNA and as long as bases thereof can be complementarily bound to bases of the terminal side sequence.

Similarly, the RNA analogues are not particularly limited as long as they can be complementarily bound to the terminal side sequence. More specifically, the RNA analogues may be used without any particular limitation as long as they are obtained by modifying a phosphate main chain, ribose, and/or a base selected from adenine, guanine, cytosine, and uracil of RNA and as long as bases thereof can be complementarily bound to bases of the terminal side sequence.

For example, the phosphate main chain may be modified by chemical modification such as phosphorothioation. The deoxyribose and ribose (hereinafter, sometimes generically referred to as “pentose”) may be modified by, for example, chemical modification such as o-methylation. Examples of nucleic acid analogues whose phosphate main chain and pentose have been modified include peptide nucleic acids.

The oligonucleotide has the same base length as that of the predetermined terminal side sequence. That is, the oligonucleotide preferably has a length of 15 to 40 bases, more preferably a length of 20 to 30 bases. If the base length of the oligonucleotide is less than the above lower limit value, there is a case where annealing with mRNA cannot be carried out. On the other hand, even if the base length of the oligonucleotide exceeds the above upper limit value, the same effect can be obtained. However, from the viewpoint of production cost, it is preferable that the base length of the oligonucleotide does not exceed the above upper limit value or so.

It is to be noted that as described above, terminal side sequences having different base lengths may be selected from one terminal region, and therefore oligonucleotides having different base lengths may be used per one terminal region.

Particularly, in the present invention, in a case where a poly(A) chain sequence or a partial sequence thereof is selected as the terminal side sequence, a simple sequence, such as an oligomer of thymidylic acid or an oligomer of uridylic acid, can be used as an oligonucleotide complementary to the terminal side sequence. Since mRNA often has a poly(A) chain sequence, an oligomer of thymidylic acid or an oligomer of uridylic acid can be used as a versatile oligonucleotide.

The oligonucleotide to be used in the present invention may be prepared by using, for example, a method used for primer synthesis in PCR, without any particular limitation.

The oligonucleotide may be used in such a manner that the concentration thereof in the reaction solution is in the range of 0.1 to 5 μM, preferably in the range of 0.5 to 1.5 μM. If the concentration of the oligonucleotide in the reaction solution is less than the above lower limit value, addition of the oligonucleotide produces little effect. On the other hand, if the concentration of the oligonucleotide in the reaction solution exceeds the above upper limit value, there is a case where the amount of synthesized protein is reduced rather than increased.

As has been described above, according to the present invention, the amount of synthesized protein can be increased by simply using the oligonucleotide. Therefore, a large amount of protein can be synthesized without using a method for suppressing the degradation of mRNA from the end thereof, such as addition of a cap structure or preparation of looped mRNA. The mechanism for increase in the amount of synthesized protein is not exactly known, but it can be considered that annealing of the oligonucleotide with the terminal side sequence of mRNA suppresses the degradation of mRNA from the end thereof.

The reaction temperature of protein synthesis is usually in the range of 10 to 40° C., preferably in the range of 15 to 30° C. If the reaction temperature is less than 10° C., the rate of protein synthesis tends to be lower. On the other hand, if the reaction temperature exceeds 40° C., components essential for protein synthesis tend to be denatured.

In the present invention, cell-free protein synthesis may be carried out by a batch method. The reaction time of protein synthesis is not particularly limited, and may be appropriately determined by those skilled in the art. As a guide, the reaction time may be set to about 6 hours.

The amount of protein synthesized according to the cell-free protein synthesis method of the present invention may be measured by, for example, enzyme activity assay, SDS-PAGE, or immunoassay.

The cell-free protein-synthesis method of the present invention can be applied to protein synthesis without any particular limitation.

As has been described above, according to method of the present invention, the amount of synthesized protein can be increased by simply using the oligonucleotide. The oligonucleotide to be used in the present invention can be prepared by various methods which have been already established and practically used per se. Therefore, it can be said that the method of the present invention is a more practical method by which protein can be more easily synthesized at lower cost. Further, one of the oligonucleotides usable in the present invention is an oligonucleotide complementary to a poly(A) chain sequence (e.g., an oligomer of thymidylic acid, or an oligomer of uridylic acid), and such an oligonucletide can be more easily prepared as compared to other oligonucleotides. Furthermore, since mRNA often has a poly(A) chain sequence at the 3′ end thereof, it can be said that the method of the present invention is more versatile.

Moreover, according to the present invention, it is possible to synthesize a large amount of protein without using a method for suppressing the degradation of mRNA from the end thereof, such as addition of a cap structure or preparation of looped mRNA. From such a viewpoint, it can be said that the method of the present invention is a more practical method by which protein can be more easily synthesized at lower cost.

### EXAMPLES

The present invention will be explained in further detail by way of examples, however, the present invention is not limited to these examples.

Green fluorescent protein (GFP) was synthesized in a cell-free protein synthesis system using an extract solution derived from High Five cells (manufactured by Invitrogen) as insect cells.

In Examples 1 and 2 and Comparative Example 1, the following oligonucleotides were used. More specifically, as an oligonucleotide complementary to a 5′ terminal side sequence, an oligonucleotide (ASO1) complementary to a sequence (having a length of 30 bases) present in an untranslated region located upstream of an Ω sequence was used, and as an oligonucleotide complementary to a 3′ terminal side sequence, an oligonucleotide (ASO2) complementary to a partial sequence (having a length of 30 bases) of a poly(A) chain sequence was used. These base sequences are as follows:

(i) ASO1

(SEQ ID No:1)

5′-AATTAAAAATAAAAACGCGAATTCCGTGTA-3′
(ii) ASO2

(SEQ ID No:2)

5′-TTTTTTTTTTTTTTTTTTTTTTTTTTTTTT-3′

### Example 1

An insect cell extract solution was prepared according to the following method. The method is based on the method of Example 1 disclosed in Japanese Unexamined Patent Publication No. 2004-215651.

<Culture of Insect Cells>

2.1×10⁷ High Five insect cells (manufactured by Invitrogen) were cultured in a culture flask (600 cm²) containing an Express Five serum-free medium (manufactured by Invitrogen) supplemented with L-glutamine at 27° C. for 6 days. As a result, the number of cells was increased to 1.0×10⁸, and the wet weight of the cells was 1.21 g.

<Preparation of Insect Cell Extract Solution>

First, the cultured insect cells were collected, and were then washed (centrifugation at 700×g at 4° C. for 10 min) 3 times with a solution for extraction having the following composition. The washed cells were suspended in 1 mL of the solution for extraction.

[Composition of Solution for Extraction]

| | | |
|---|---|---|
| 60 | mM | HEPES-KOH (pH 7.9) |
| 200 | mM | potassium acetate |
| 4 | mM | magnesium acetate |
| 4 | mM | DTT |
| 0.5 | mM | PMSF |

The thus obtained suspension was rapidly (within 10 sec) frozen in liquid nitrogen. After being sufficiently frozen, the suspension was thawed in a water bath at about 10° C. After being thawed completely, the suspension was centrifuged at 15,000×g at 4° C. for 15 min (“himacCR20B3” manufactured by Hitachikoki Co., Ltd.) to collect supernatant. 1.5 mL of the collected supernatant was applied to a PD-10 desalting column (manufactured by Amersham Bioscience) equilibrated with a buffer solution for gel filtration having the following composition.

[Composition of Buffer Solution for Gel Filtration]

| | | |
|---|---|---|
| 40 | mM | HEPES-KOH (pH 7.9) |
| 100 | mM | potassium acetate |
| 2 | mM | magnesium acetate |
| 1 | mM | DTT |
| 0.5 | mM | PMSF |

After being applied to the desalting column, the supernatant was eluted with 4 mL of the buffer solution for gel filtration, and then fractions having an absorbance at 280 nm of 30 or more were collected using a spectrophotometer (“Ultrospec3300pro” manufactured by Amersham Bioscience). The collected filtrate was further centrifuged at 45,000×g at 4° C. for 30 min to collect supernatant. The thus obtained supernatant was used as an insect cell extract solution.

<Preparation of Exogenous mRNA>

According to the protocol of PROTEIOS (TOYOBO), a plasmid pEU-GFP was constructed by inserting a green fluorescent protein (GFP) gene into the downstream of an Ω sequence of a vector pEU (manufactured by TOYOBO), and then mRNA was synthesized using a circular DNA (it is to be noted that mRNA synthesis may also be carried out by using a PCR product obtained using the circular DNA as a template). According to the protocol of RiboMAX Large Scale RNA Production System (Promega), ethanol precipitation and gel filtration were carried out after the completion of mRNA synthesis, and then the amount of synthesized mRNA was measured by ultraviolet absorption spectrometry. The thus obtained mRNA was used for cell-free protein synthesis.

<Cell-Free Protein Synthesis>
[Composition of Reaction Solution]

| | | |
|---|---|---|
| 40 | mM | HEPES-KOH (pH 7.9) |
| 100 | mM | potassium acetate |
| 2 | mM | magnesium acetate |
| 0.5 | mM | ATP |
| 0.25 | mM | GTP |
| 20 | mM | creatine phosphate |
| 200 | μg/mL | creatine kinase |
| 2 | mM | DTT |
| 80 | μM | amino acid (20 kinds) |
| 0.25 | mM | EGTA |
| 200 | μg/mL | tRNA (derived from brewer's yeast) |
| 2 | U/μL | RNase inhibitor |
| 50% | (v/v) | extract solution |
| 320 | μg/mL | mRNA |
| 1.25 | μM | oligonucleotide (above-mentioned ASO2) |

The total amount of the reaction solution having the above composition was 40 μL (that is, the amount of mRNA contained in the reaction solution was 12.8 μg). Protein synthesis was carried out using the reaction solution at 25° C. for 6 hours.

### Example 2

Protein synthesis was carried out in the same manner as in the Example 1 except that the ASO2 as oligonucleotide was replaced with an 1:1 (weight ratio) mixture of ASO1 and ASO2 so that the reaction solution contained 1.25 μM of the mixture.

### Comparative Example 1

Protein synthesis was carried out in the same manner as in the Example 1 except that the ASO2 as oligonucleotide was replaced with the above ASO1.

### Comparative Example 2

Protein synthesis was carried out in the same manner as in the Example 1 except that the oligonucleotide was not used.

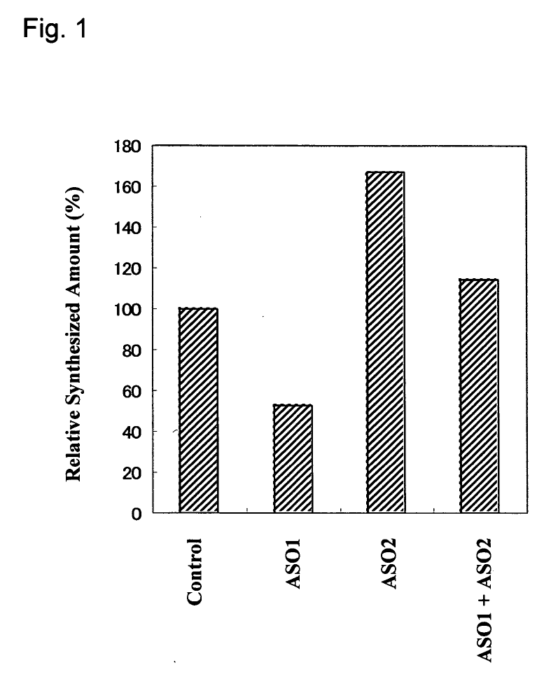

For each of the GFPs synthesized in the Examples 1 and 2 and the Comparative Examples 1 and 2, fluorescence intensity was measured using a microplate reader GENios (manufactured by TECAN). The concentration of the synthesized GFP was determined using a GFP concentration-fluorescence intensity calibration curve prepared by measuring the fluorescence intensity of each dilute solution having a known GFP concentration of a GFP dilution series. FIG. 1 is a bar graph showing a comparison of the amount of GFP synthesized by the method of the present invention (Examples 1 and 2) or by a reference method (Comparative Example 1) to the amount of GFP synthesized by a conventional method (Comparative Example 2). In FIG. 1, the axis of ordinate represents a relative amount of synthesized GFP (%), and the result obtained by the conventional method (Comparative Example 2) is indicated by a bar “Control”, the result obtained by the method of the present invention using ASO2 as oligonucleotide (Example 1) is indicated by a bar “ASO2”, the result obtained by the method of the present invention using ASO1 and ASO2 as oligonucleotide (Example 2) is indicated by a bar “ASO1+ASO2”, and the result obtained by the reference method using ASO1 as oligonucleptide (Comparative Example 1) is indicated by a bar “ASO1”. As can be seen from FIG. 1, the amount of synthesized GFP was increased in either case according to the method of the present invention. The amount of synthesized GFP was increased in the following order: Comparative Example 1<Control (Comparative Example 2)<Example 2<Example 1. The maximum amount of synthesized GFP was about 1.7 times the amount of GFP synthesized by the conventional method (Comparative Example 1).

The above-described Examples show concrete modes within the scope of the present invention, however, the present invention can be carried out in various other modes. Therefore, the above-described Examples are merely illustrative in all respects, and must not be construed as being restrictive. Further, the changes that fall within the equivalents of the claims are all within the scope of the present invention.

What is claimed is:

1. A method for cell-free protein synthesis performed in a reaction solution containing mRNA and a living cell-derived extract solution, the reaction solution containing an oligonucleotide complementary to a sequence present in a 3′ terminal region of the mRNA.

2. The method for cell-free protein synthesis according to claim 1, wherein the reaction solution further contains an oligonucleotide complementary to a sequence present in a 5′ terminal region of the mRNA.

3. The method for cell-free protein synthesis according to claim 1, wherein the oligonucleotide is selected from the group consisting of DNA, DNA alanogues, RNA, and RNA analogues.

4. The method for cell-free protein synthesis according to claim 1, wherein the sequence present in the 3′ terminal region of the mRNA is a poly(A) chain sequence.

5. The method for cell-free protein synthesis according to claim 1, wherein the mRNA has a translation enhancer sequence at a position adjacent to a downstream side of the 5′ terminal region.

6. The method for cell-free protein synthesis according to claim 1, wherein the oligonucleotide has a length of 15 to 40 bases.

7. The method for cell-free protein synthesis according to claim 1, wherein a concentration of the oligonucleotide contained in the reaction solution is in a range of 0.1 to 2 μM.

8. The method for cell-free protein synthesis according to claim 1, wherein the living cell is an insect cell.


SEQUENCE LISTING (2 sequences)

SEQ ID NO: 1 - DNA, 30 bp, Artificial Sequence
  Description of Artificial Sequence synthetic oligonucleotide
aattaaaaataaaaacgcgaattccgtgta

SEQ ID NO: 2 - DNA, 30 bp, Artificial Sequence
  Description of Artificial Sequence synthetic oligonucleotide
tttttttttttttttttttttttttttttt